(19) United States
(12) Patent Application Publication
(10) Pub. No.: US 2005/0123906 A1
(43) Pub. Date: Jun. 9, 2005

(54) Protein modulation

(76) Inventor: Tariq M. Rana, Shrewsbury, MA (US)

Correspondence Address: FISH & RICHARDSON PC, 225 FRANKLIN ST, BOSTON, MA, 02110, US

(21) Appl. No.: 10/984,946

(22) Filed: Nov. 8, 2004

Related U.S. Application Data
(60) Provisional application No. 60/518,543, filed on Nov. 6, 2003.

(51) Int. Cl. C12Q 1/70; A61K 31/5513; A61K 31/53; C12Q 1/68

(52) U.S. Cl. 435/5; 514/221; 514/241; 435/6

(57) ABSTRACT

The present invention provides methods for screening and identifying compounds that inhibit a pathway affecting protein levels, and methods and compounds for treating viral, e.g., HIV, infection.

### CLAIM OF PRIORITY

This application claims the benefit under 35 USC §119(e) of U.S. Provisional Patent Application Serial No. 60/518,543, filed on Nov. 6, 2003, the entire contents of which are hereby incorporated by reference.

### TECHNICAL FIELD

This invention relates to methods of identifying compounds that inhibit protein function.

### BACKGROUND

The etiology of many human diseases and disorders has been traced to the activity of one protein on a second protein, e.g., to limit or reduce levels of the second protein.

### SUMMARY

Described herein are simple and yet elegant methods to screen and identify compounds that inhibit pathways in protein functions. The mechanism of protein function need not be fully known to perform the methods. The methods described herein can be used to screen for modulators of pathways involving, for example, mRNA turnover as well as message translocation or transcription inhibition, or protein turnover, translocation, or translation inhibition. One example of such a screen is described herein and is used to identify inhibitors of a protein called Vif that is involved in HIV replication, but this technology has wide applicability to screen for modulators of almost any protein-protein interaction pathway wherein the level of one of the protein interactants is affected. Additional examples include LacI and LacZ (Beckwith, Science (1967) 156(3775):597-604); HMG box-containing protein 1 (HBP1) and the growth regulatory genes it suppresses (e.g., N-Myc, c-Myc, and cyclin D1), or p47phox, a subunit of NADPH oxidase (Berasi et al., Mol Cell Biol. (2004) 24(7):3011-24); p53 and the genes it regulates, including cyclin-dependent kinase inhibitor p₂₁^{WAF-1}, 14-3-3, reprimo, and genes involved in the apoptotic cascade, e.g., bax, DR5, p53AIP, PIDD, NOXA, PUMA, Fas/APO-1 and redox related genes; and Mdm2 and p53 (Alarcon-Vargas and Ronai, Carcinogenesis (2002) 23(4):541-547). As a number of these interactions are important in disease processes including viral infection and cancerous cell proliferation, the methods described herein can be used to identify potential therapeutic compounds for the treatment of diseases associated with the regulation of the second protein by the first protein.

Thus, the present invention provides methods for screening and identifying compounds that inhibit a pathway affecting protein levels. These methods can be utilized to identify small molecule or other inhibitors of protein function for any protein that affects the level of a second protein. Described herein are general methods for screening compound libraries, e.g., libraries of small molecules, to identify compounds that inhibit the activity of a protein.

In one aspect, the invention features methods for identifying an modulator of an activity of a first protein, wherein the activity of the first protein modulates a level of a second protein, e.g., normally modulates the level of the second protein. “normally” can include modulation that occurs under physiologically healthy conditions, and modulations that occur under disease conditions. The methods include obtaining a sample, e.g., a sample comprising a cell expressing a first tagged protein and a second tagged protein, or a cell lysate or cell-free extract including a first tagged protein and a second tagged protein; contacting the sample with a test compound; determining a level of a first protein and a level of a second protein in the presence of the test compound; determining a test ratio of the levels of the first and second proteins in the presence of the test compound; and obtaining a reference ratio of a level of the first and second proteins in the absence of the test compound. A change in the test ratio compared to the reference ration indicates that the test compound is a modulator of the activity of the first protein. The activity of the first protein can cause a reduction or an increase in the level of the second protein, e.g., by affecting one or more of transcription, translation, sub-cellular localization, degradation, or post-translational modification of the second protein.

In some embodiments, the first and second tagged proteins each have fluorescent tags that are excited at different wavelengths, emit at different wavelengths, or both. The fluorescent tags can be, e.g., green fluorescent protein, yellow fluorescent protein, red fluorescent protein, cyan fluorescent protein, Kindling red protein, and JRed.

In some embodiments, the cell or sample further expresses or includes a third tagged protein; in this case, the first, second, and third tagged proteins (or a subset thereof) can each comprise fluorescent tags that are excited at different wavelengths, emit at different wavelengths, or both.

In some embodiments, the first protein is Virion Infectivity Factor (Vif) and the second protein is apolipoprotein B mRNA-editing enzyme, catalytic polypeptide-like 3G (APOBEC3G).

In some embodiments, the first protein is p53 and the second protein is selected from the group consisting of cyclin-dependent kinase inhibitor p21WAF-1, 14-3-3, reprimo, bax, Death receptor 5 (DR5), p53-regulated apoptosis-inducing protein 1 (p53AIP), p53 Protein Induced with Death Domain (PIDD), NOXA, p53 Upregulated Modulator of Apoptosis (PUMA), Fas/Apoptosis Inducing Protein 1 (Fas/APO-1), and by redox related proteins.

In some embodiments, the first protein is Mdm2 and the second protein is p53.

In another aspect, the invention features the small molecules described herein, e.g., small molecule modulators, e.g., Vif inhibitors identified by a method described herein, and compositions including one or more small molecule inhibitors of Virion Infectivity Factor (Vif) and a pharmaceutically acceptable carrier.

In a further aspect, the invention provides methods of treating subjects infected with a virus, e.g., a lentivirus, e.g., HIV. The methods include administering to the subject a therapeutically effective amount of a composition described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a reproduction of a Western blot of protein levels of Cyan Fluorescent Protein (CFP)-APOBEC3G and Yellow Fluorescent Protein (YFP)-Vif fusion proteins, 24 hours after co-transfection in increasing molar ratios of expression vectors into 293T cells at the specified ratios (value of 1=130 fmoles).

FIGS. 1B-1D are graphs that illustrate the results of fluorometric analysis 24 hours after co-transfection of expression vectors into 293T cells at the specified ratios (value of 1=130 fmoles). FIG. 1B, CFP-APOBEC3G co-transfected with increasing molar ratios of YFP-Vif; FIG. 1C, CFP-APOBEC3G co-transfected with increasing molar ratios of NL4GFP-HIV proviral DNA. Virus production was measured by virus-derived Green Fluorescent Protein (GFP). FIG. 1D, cells co-transfected with a constant amount of pNL-AlΔvif or pNL-A1 and a range of pCFP-APO. The molar ratio of each vector (pHIV:pAPO) is presented (1=130 fmoles). Cells were treated with ALLN (+) or the equivalent volume of DMSO (−) for 12 hours prior to preparation of total cell lysates 36 hours post-transfection. Proteins were visualized by immuno-blot analysis of CFP-APO (α-GFP) and Vif (α-Vif). Endogenous CycT1 (α-CycT1) was probed as a loading control.

FIG. 2A is a composite of Western blots of CFP-APOBEC3G and YFP-Vif co-transfected into 293T cells at a 1:4 molar ratio (1=130 pmoles). At 24 hours post-transfection (time-point [TP] 0), a DMSO control, or one of the proteasome inhibitors lactacystin (10 mM), ALLN (150 mM), or MG-132 (10 mM) was added to growth media as indicated. Cells were then harvested at various time points up to 12 hours after addition of inhibitor and total cell lysates were analyzed with a-GFP to detect both CFP-APOBEC3G and YFP-Vif. As a control for protein loading, endogenous CycT1 levels were observed at TPs 0 and 12 (right hand column). As a control for proteasome inhibition, the levels of endogenous P27 were observed at TPs 0 and 12 (right hand-column). CFP-APO3G=CFP-APOBEC3G

FIG. 2B is a composite of a gel showing the results of real-time PCR performed on oligo d(T) primed cDNA using primers that amplified the entire coding region of either APOBEC3G (AP03G) or Vif. CFP-APOBEC3G and YFP-Vif vectors were co-transfected into 293T cells at a 1:8 molar ratio (1=130 pmoles), respectively, and total m-RNA was harvested from cells 24 hours post-transfection. To control for DNA contamination, the same reactions lacking reverse transcriptase (-RT) were included.

FIG. 3A is a Western blot of protein isolated from cells co-transfected with YFP-Vif and vectors that express CFP, CFP-APOBEC3G or APOBEC3G-CFP, all at a 1:8 molar ratio (1=130 pmoles), probed with an anti-GFP antibody.

FIG. 3B is a schematic illustration of an APOBEC3G-CFP vector designed to promote translation of both full length APOBEC3G-CFP and CFP alone from the same mRNA transcript, through the addition of a Kozak sequence (CCACC) and start codon upstream of the CFP coding region.

FIG. 4 is a schematic illustration of one embodiment of a high-throughput screening method described herein.

FIGS. 5A-5C are schematic illustrations of one embodiment of a high-throughput screening method described herein. FIG. 5A, 293T Cells expressing only YFP-Vif. 5B, 293T Cells expressing the target protein, APOBEC3G 5C, 293T cells expressing YFP-Vif, CFP-ABOBEC3G or both are cultured in a 96 well plate. After treating each well of cells with a different small molecule, a fluorimeter can be used to screen the 96-well plate for cells emitting increased CFP fluorescence. \mathring{\}, small molecules

FIGS. 6A-6C are exemplary bar graphs showing that cells containing both YFP-Vif and CFP-Apo are expected to show reduced CFP fluorescence, but that the addition of a small molecule (6C) will cause a recovery of CFP fluorescence.

FIG. 7 is a bar graph showing CFP fluorescence in a CFP-APOBEC3G/YFP-Vif bioassay in a 96 well plate. Again, cells containing both YFP-Vif and CFP-Apo show reduced CFP fluorescence.

FIG. 8 is a schematic illustrating the structures of several small molecule inhibitors of Vif identified by a method described herein.

### DETAILED DESCRIPTION

The methods described herein can be used to identify modulators, e.g., small molecules or other molecules, that alter the effect of one protein on levels of a second protein. The methods typically include expressing two fluorescent fusion proteins, e.g., proteins which include a fluorescent tag linked to a non-fluorescent protein. The fusion proteins are expressed in the same cell, and the fluorescence levels of each are measured. Typically, the two fusion proteins include a first protein that has an effect on the level of the second protein; for example, the first protein may reduce levels of the second protein, e.g., by affecting some part of the synthesis or degradation pathway of the second protein. In this case, in the absence of any modulators of the activity of the first protein, the fluorescence emitted by the tag of the second protein will be low. In the presence of an inhibitor, the fluorescence of the second tag will increase, e.g., relative to the fluorescence of the first tag. In the presence of an enhancer, the fluorescence of the second tag may decrease further. The methods are also applicable where the first protein increases levels of the second protein.

Using this method in a model system including Vif as the first protein and APOBEC3G as the second protein, several inhibitors of Vif's activity on APOBEC3G levels were identified.

Screening Methods

The invention includes methods for identifying modulators, e.g., compounds such as small molecules, of the activity of a first protein on a second protein, wherein the levels (i.e., concentrations or amounts) of the second protein are modulated (e.g., reduced or increased) by the activity of the first protein. The methods include the expression of two fusion proteins in a cell culture system, e.g., including a first protein known to have an effect on the level of a second protein. Each protein is linked to an easily detectable tag, e.g., a fluorescent protein tag (including, but not limited to, Green Fluorescent Protein (GFP), Blue Fluorescent Protein (BFP), Red Fluorescent Protein (RFP), Yellow-Fluorescent Protein (YFP), or Cyan Fluorescent Protein (CFP), monomeric RFP (mRFP) (Campbell et al., 2002 ), cyan fluorescent protein (CFP), JRed (Shagin et al., Mol. Biol. Evol. (2004) 21(5):841-850), Kindling Red (Chudakov et al., Nature Biotechnology (2003) February;21(2):191-194) or an enhanced version thereof), such that the first and second proteins are expressed with tags that are excited at and/or emit at different wavelengths. In some embodiments, the first tag is YFP and the second tag is CFP, or vice-versa. In some embodiments, the tag is an epitope tag, e.g., a myc tag, a histidine (His) tag, or a hemagglutinin (HA) tag. A number of suitable tags are known in the art, and many are commercially available. The levels of each protein are detected by detecting the levels of the tags. Typically, the level of the first protein can be used as an internal control, and the ratio of the levels of the first and second proteins determined. An increase in the level of the second protein can thus be determined as an increase in the level of the tag on the second protein relative to the level of the tag on the first protein; a decrease can be determined similarly.

In some embodiments, e.g., where the first protein acts to regulate the level of more than one other protein, other proteins regulated by the first protein are also tagged, and their levels are evaluated. In this way, selective compounds that modulate only the effect of the first protein on the second protein, and not any effect on a third protein, can be identified. The methods can be adapted to identify compounds that affect the activity of the first protein on a set of selected proteins, but not on other proteins. In some embodiments, the proteins (or subsets thereof) are tagged with fluorophores that are excited at and/or emit at different wavelengths.

A number of methods are known in the art that can be used for producing such fusion constructs. For example, a number of vectors including the sequence of suitable tags are commercially available (e.g., from Becton, Dickinson and Company/Clontech, Palo Alto, Calif.; additional information, including sequences, can be obtained from the company, e.g., on their website) or can be produced using known methods in the laboratory; sequences encoding the first and second proteins can be cloned into the vector such that the sequence of the protein is in frame with the tag, and transfection of the vector into a competent host cell will result in the expression of a fusion protein comprising the protein and the tag. In some embodiments, the tag is at the N-terminal end of the protein; in some embodiments, the tag is at the C-terminal end of the protein. In some embodiments, the sequences encoding the fusion proteins includes a promoter or other regulatory sequence native to the first and/or second proteins. Preferably, the tag does not interfere with the activity of the first and/or second protein, and does not interfere with the activity of the first protein on the second protein.

The fusion proteins can then be expressed together in cultured cells using methods known in the art, and protein levels monitored, e.g., by monitoring the levels of the tags. One of skill in the art will appreciate that the method to be used for monitoring the levels of the tags will depend on the tag selected, e.g., fluorescence microscopy or flow cytometry for fusion proteins including a fluorescent tag. Where the tag does not itself fluoresce (e.g., an epitope tag), the levels of the tag can be monitored, e.g., using a fluorescent-tagged primary or secondary antibody. In cells expressing both of the constructs, the presence of the first protein will modulate the levels of the second protein, thus modulating the levels of the second tag, e.g., a fluorescent tag that is easily detectable.

In some embodiments, libraries of test compounds, e.g., small molecules, are screened by observing the levels of the second tagged protein in the absence and the presence of the first tagged protein, and in the absence and the presence of a test compound, e.g., a small molecule candidate inhibitor. In some embodiments, the methods include the use of high-throughput screening methods as known in the art, e.g., using plates having multiple, e.g., 96, or more, wells. Typically, the method of detection involves standard or direct detection of fluorescence emitted by the fluorescent tag, i.e., not a fluorescence resonance energy transfer (FRET)-based or fluorescence polarization (FP)-based method.

Libraries of test compounds, such as small molecules, are available, e.g., commercially available, or can be synthesized using methods known in the art. As used herein, “small molecules” refers to small organic or inorganic molecules. In some embodiments, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da). The compounds can include organic or inorganic naturally occurring or synthetic molecules including but not limited to soluble biomolecules such as oligonucleotides, polypeptides, polysaccharides, antibodies, and fatty acids.

The test compounds can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecule compounds are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czamik, (1997) Curr. Opin. Chem. Bio. 1:60).

In some embodiments, the test compounds are peptides or peptidomimetics, e.g., of about 300 Da to about 12,000 Da. As used herein, a “peptidomimetic” is a synthetic compound that is structurally similar to a peptide, but contains non-peptidic structural elements. A peptidomimetic lacks at least one classical peptide characteristic, such as enzymatically scissile peptidic bonds. For example, a peptidomimetic can have an unnatural backbone, e.g., oligocarbamate (with a carbamate-containing backbone); oligourea (with a urea-containing backbone), e.g., as described in Tamilarasu et al. (2000) Bioorg. Med. Chem. Lett. 10(9):971-4; Tamilarasu et al. (1999) J. Am. Chem. Soc. 121:1597-1598; oligothiourea (with a thiourea-containing backbone); and N-substituted peptoids (peptidomimetics that results from the oligomeric assembly of N-substituted amino acids), e.g., oligopeptoid ester or amide analogs, e.g., as described in Kesavan et al., (2002) Bioconjug. Chem. 13(6):1171-5.

In some embodiments, the test compounds include, but are not limited to, peptide analogs including peptides comprising non-naturally occurring amino acids, e.g., β-amino acids or β-substituted β-amino acids (“β³-amino acids”), phosphorous analogs of amino acids, such as α-amino phosphonic acids and α-amino phosphinic acids, or amino acids having non-peptide linkages, or other small organic molecules. In some embodiments, the compounds are β-peptide molecules; peptidomimetics (e.g., peptoid oligomers, e.g., peptoid amide or ester analogues, β-peptides, D-peptides, L-peptides, oligourea or oligocarbamate); small peptides (e.g., tripeptides, tetrapeptides, pentapeptides, or larger); cyclic peptides; other non-natural or unnatural peptide-like structures; and inorganic molecules (e.g., heterocyclic ring molecules). In some embodiments, the test compounds are nucleic acids.

In one embodiment, the test compounds are β-peptides. The study of β-peptides has accelerated over the past decade, propelled by demonstrations that they can be programmed to adopt protein-like secondary structures. These structures have given rise to a variety of biological activities, and the protease resistance of P-peptides makes them attractive from a pharmaceutical standpoint.

Libraries screened using the methods described herein can comprise a variety of types of compounds. A given library can comprise a set of structurally related or unrelated test compounds. In some embodiments, the compounds and libraries thereof can be obtained by systematically altering the structure of a first compounds, e.g., small molecule, e.g., a first small molecule that is structurally similar to a known natural binding partner of the first or second protein, or a first small molecule identified as capable of binding the first or second protein, e.g., using methods known in the art or the methods descried herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

Test compounds identified as “hits” (e.g., small or other molecules that cause an increase in the second fluorescence signal in the presence of the first fusion protein, relative to a reference, e.g., the signal in the presence of the first fusion protein, in the absence of the test compound) in the first screen can be selected and systematically altered, e.g., using rational design, to optimize binding affinity, avidity, specificity, or other parameter. Optimized compounds can also be screened using the methods described herein. Thus, in one embodiment, the invention includes screening a first library of compounds using the methods described herein, identifying one or more hits in that library, subjecting those hits to systematic structural alteration to create additional libraries of compounds structurally related to the hit, and screening the second library using the methods described herein. In another embodiment, the invention includes screening a first library of compounds using a method known in the art, identifying one or more hits in that library, subjecting those hits to systematic structural alteration to create a second library of compounds structurally related to the hit, and screening the second library using the methods described herein.

The methods described herein can be used to identify modulators of protein interactions, regardless of where in the protein biosynthetic pathway the interaction takes place. This means that it does not matter where in the pathway the first protein is acting to affect the level of the second protein, e.g., whether the first protein is affecting, for example, mRNA transcription, translocation, or half-life, or protein translation, translocation, sorting, or half-life.

Methods of Identifying Virion Infectivity Factor (Vif) inhibitors

HIV-1 infectivity is highly dependent on the viral-encoded gene Vif. Vif was implicated in HIV-1 infectivity due to the varied response different types of human cells had to HIV-1 lacking Vif. Some cell types infected with HIV-1 lacking Vif still produce infectious virus and are called permissive cell types. In other cell types, designated non-permissive, HIV-1 encoding Vif can produce infectious virus while HIV-1 lacking Vif cannot. This suggested that there was a protein or subset of proteins present in non-permissive cells that Vif must neutralize for infectious HIV-1 virus to be produced. One protein, called “apolipoprotein B mRNA-editing enzyme, catalytic polypeptide-like 3G” (APOBEC3G) (previously named Cem15), has been discovered that can cause cells to become immune to HIV-1 lacking Vif (i.e., they become non-permissive) (Madani and Kabat, (2000) J. Virol. 74:5982-5987). As a DNA editing enzyme, APOBEC3G severely mutates newly made viral cDNA, which is DNA synthesized from viral RNA during HIV-1 reverse transcription (Gu and Sundquist, (2003) Nature 424:21-22). In the absence of Vif, APOBEC3G is packaged with the virus and exerts its effect after the virus infects another host cell. Highly mutating viral cDNA during the early stages of reverse transcription leads to destruction of the HIV-1 genome and a detrimentally high mutation rate within genes encoded by the HIV-1 genome (Gu et al., supra). Thus, APOBEC3G has damaging effects on HIV-1 that are prevented by the presence of Vif during HIV-1 infection.

As described herein, Vif counteracts apolipoprotein B mRNA-editing enzyme, catalytic polypeptide-like 3G (APOBEC3G) by reducing the amount of APOBEC3G protein normally synthesized in cells. With less APOBEC3G protein made, less APOBEC3G protein is packaged into new viruses, and fewer viruses are affected by APOBEC3G mutating activity during the next round of infection. Based on this information, screening methods were developed for isolating compounds such as small molecules that can inhibit Vif. Using these methods, several small molecules have been identified that block Vif function. Blocking Vif function with these molecules restores normal levels of protein synthesis in cells, including synthesis of APOBEC3G and other potential host defense proteins, enhancing or providing the host response to viral infection. Thus, the present invention provides methods of identifying inhibitors, e.g., small molecule inhibitors, of Vif function, and the inhibitors identified thereby.

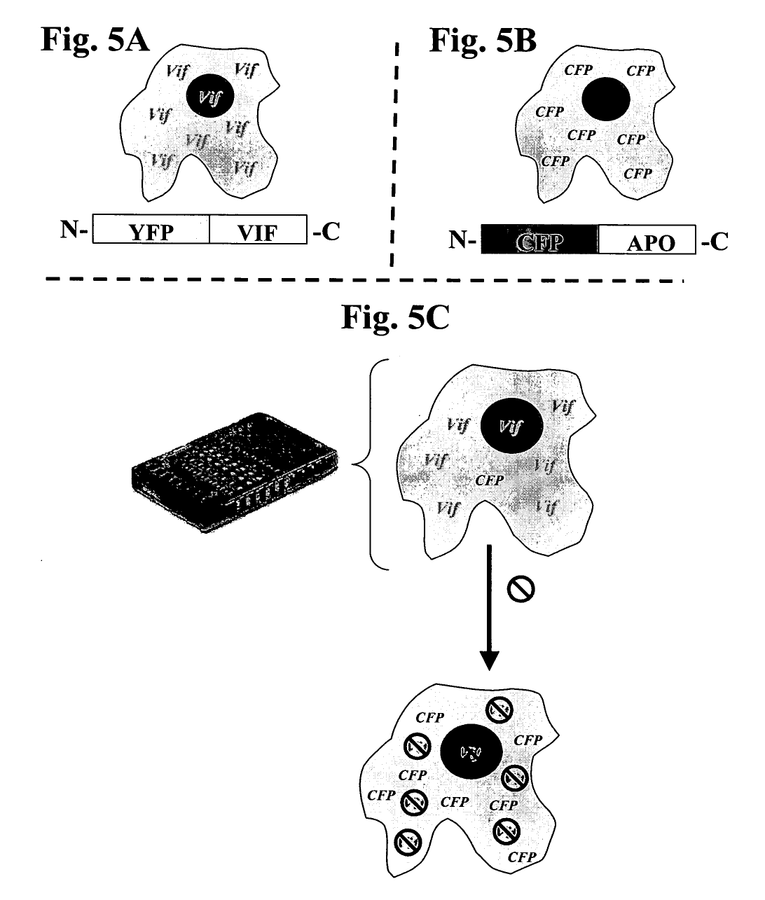

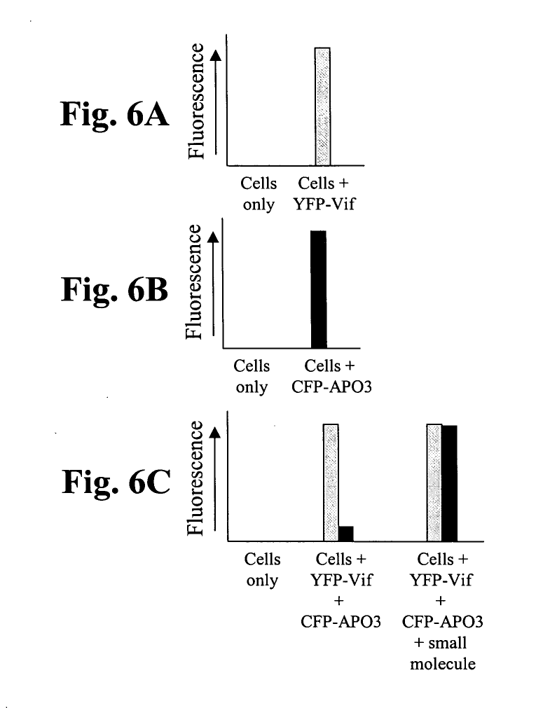

In one embodiment of the new method, 293T cells expressing a Vif fusion protein comprised of Vif linked to a-first fluorescent protein tag (e.g., an easily detectable tag; in some embodiments, this tag is the Yellow-Fluorescent Protein (YFP)) are observed (FIG. 5A and FIG. 6A), as are cells that synthesize a second fluorescent protein (e.g., another easily detectable tag, Cyan Fluorescent Protein (CFP)) linked to APOBEC3G (FIG. 5B and FIG. 6B). In addition, some cells that concomitantly synthesize both YFP-Vif and CFP-APOBEC3G are observed (FIG. 5C and FIG. 6C), e.g., in the presence or absence of a candidate small molecule inhibitor.

To assay whether the first and second fusion proteins are being synthesized in the screen, both YFP-Vif and CFP-APOBEC3G fluorescence is measured using a fluorimeter (FIGS. 6A-6C illustrate exemplary results). In cells lacking YFP-Vif, a high level of CFP fluorescence is easily detectable. However, in cells synthesizing YFP-Vif, CFP fluorescence is dramatically reduced, indicating that YFP-Vif negatively affects the synthesis of the CFP-linked APOBEC3G. In one embodiment comprising a high throughput screen (HTS), cells grown in a multiwell, e.g., 96 well, plate format are treated with an array of synthetic small molecule libraries and assayed for restoration of CFP fluorescence in the presence of sustained YFP fluorescence. Any small molecule that leads to the restoration of CFP fluorescence in the presence of YFP-Vif is expected to have blocked Vif function. These molecules are then considered “hits” (or “candidate compounds”) and can then be further evaluated for their potential as anti-viral agents, e.g., anti-HIV-1 drugs.

The methods described herein can be used with any pair of interacting proteins where one protein affects the level of the other protein. For example, the proteins can include (but are not limited to): Vif (or homologs thereof) and APOBEC3G (and homologs thereof); ubiquitin and a target protein marked by ubiquitin for degradation, e.g., p53 (as well as enzymes involved in ubiquitin-like modifications such as Sumoylation (SUMO, see, e.g., Muiller et al., Nat. Rev. Mol. Cell Bio, 2:202-210 (2001)), Neddylation (RubI or Nedd8), and ISGylation, mammalian homologues of APG and AUT (Ohsumi, Nat. Rev. Mol. Cell Bio. 2:211-217 (2001)), and their targets, e.g., PCNA, to screen, for example, for anticancer, antiviral, antibacterial or other agents (including treatments for Parkinson's Disease); NFκB and its partners, e.g., proteins involved in inflammation, the immune response, and cell death; CYLD and its substrates (e.g., TRAF2); protein kinase R and eukaryotic initiation factor 2 (eIF2) (e.g., to screen for improved thiazolidinediones); peroxisome proliferator-activated receptor (PPAR) and the Growth Arrest and DNA Damage-Inducible Gene 45 (GADD45), Minichromosome Maintenance Proteins, FLIP protein, or other growth-related genes, e.g., to screen for PPAR ligands or other anticancer agents; double-stranded RNA-activated protein kinase (PKR) and proteins of the Ras or Sos signaling pathways, e.g., to screen for effective anticancer and antiviral agents; and Tumor necrosis factor-related apoptosis-inducing ligand (TRAIL) and c-Jun N-terminal kinase (JNK).

A number of human and animal diseases are known which can be attributed to such arrangements, including cancers and neoplasms, viral and bacterial infections, inflammatory disorders, autoimmune disorders, and degenerative diseases, among others. Thus, the methods described herein can be used to screen libraries of compounds for potentially therapeutically useful agents for a number of diseases.

Vif Inhibitory Compounds (Vif-Is)

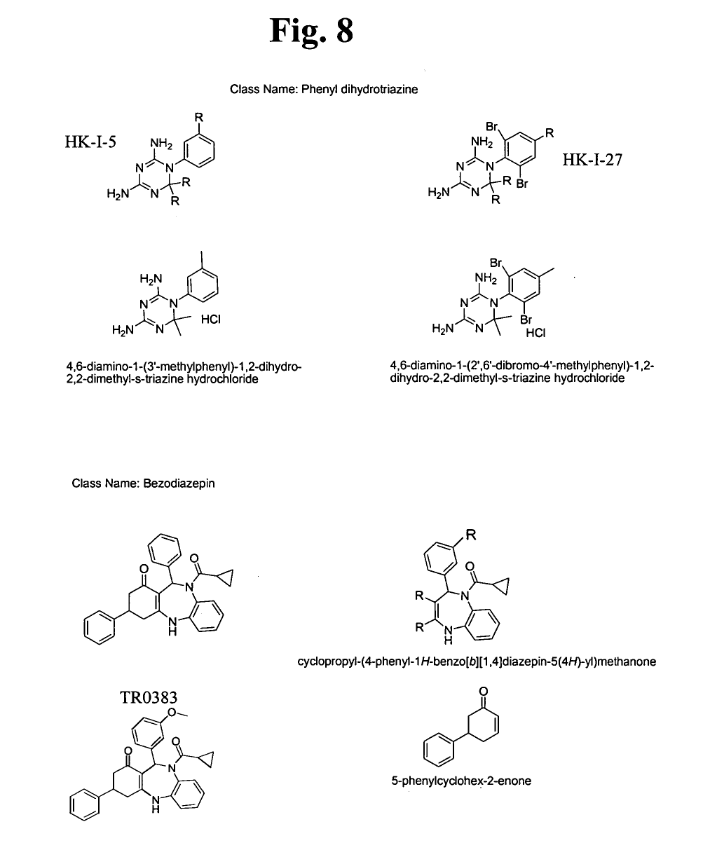

The invention also includes inhibitory compounds identified by the methods described herein. Included are Vif inhibitory compounds, or Vif-Is. The general structure of these compounds is illustrated in FIG. 8.

In some embodiments, a Vif-I molecule has the formula (I): 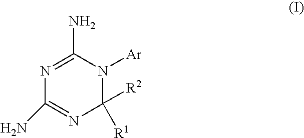
wherein
R¹ is C₁-C₆ alkyl, alkenyl, alkynyl, cycloalkyl, or cycloalkenyl, R¹ being optionally substituted with one or more of: —R^{a}, —OR^{a}, ═O, —SR^{a}, ═S, —C(═O)NR^{a}R^{b}, NR^{a}R^{b}C(═O)R^{c}, or an aryl group;
R² is C₁-C₆ alkyl, alkenyl, alkynyl, cycloalkyl, or cycloalkenyl, R² being optionally substituted with one or more of: —R^{a}, —OR^{a}, ═O, —SR^{a}, ═S, —C(═O)NR^{a}R^{b}, NR^{a}R^{b}C(═O)R^{c}, or an aryl group;
Ar is an aryl or heteroaryl group optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group; and
each R^{a}, R^{b}, and R^{c} is, independently, hydrogen, halo, substituted or unsubstituted amino, or a substituted or unsubstituted C₁-C₆ acyl, alkyl, alkenyl, alkynyl, cycloalkyl, or cycloalkenyl group.

In some embodiments, the Vif-I is HK-I-5 or HK-I-27, illustrated in FIG. 8.

In some embodiments, each R^{a}, R^{b}, and R^{c} is, independently, C₁-C₆ alkyl. In some embodiments, Ar is a 5-membered or a 6-membered aryl group. In some embodiments, Ar is a 3-substituted phenyl group. In some embodiments, Ar is 3-methoxyphenyl. In some embodiments, Ar is a 4-substituted 2,6-dihalophenyl group.

In some embodiments, a Vif-I molecule has the formula (II): 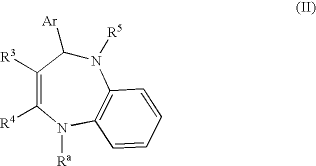
wherein
R³ and R⁴ are each, independently, C₁-C₆ alkyl, alkenyl, alkynyl, cycloalkyl, or cycloalkenyl, or R³ and R⁴ together are a C₂-C₆ alkyl or alkenyl chain;
R³ and R⁴ are, independently or together, optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group;
Ar is an aryl or heteroaryl group optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group;
R⁵ has the formula —C(═O)R^{a}; and
each R^{a}, R^{b}, and R^{c} is, independently, hydrogen, halo, substituted or unsubstituted amino, or a substituted or unsubstituted C₁-C₆ acyl, alkyl, alkenyl, alkynyl, cycloalkyl or cycloalkenyl group.

In some embodiments, R¹ and R² together are a C₂-C₆ alkyl chain. In some embodiments, Ar is unsubstituted phenyl. In some embodiments, R⁵ is an alkylcarbonyl or a cycloalkylcarbonyl group.

In some embodiments, the Vif-I is TR0383, illustrated in FIG. 8.

Vif-I Pharmaceutical Compositions and Methods of Treatment

The screening methods described herein set the groundwork for targeted development of therapeutics that inhibit specific protein activity, for example, Vif effects on the synthesis of human proteins required to fight HIV-1 infection. Vif inhibitory compounds identified using the methods described herein can be used therapeutically to restore the innate HIV-1 immunity given to cells by APOBEC3G and other human host defense proteins, thereby treating subjects having viral, e.g., HIV-1, infections. Thus, the invention also includes therapeutic compositions comprising the Vif-inhibitory (Vif-I) small molecules described herein, and methods of treatment comprising administering the Vif-Is.

The Vif-I molecules of the invention can be incorporated into pharmaceutical compositions. Such compositions typically include the Vif-I molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will typically include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the typical methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of a Vif-I compound can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The Vif-I compounds can also be prepared in the form of suppositories (e.g., with conventional suppository bases such as cocoa butter and other glycerides) or retention enemas for rectal delivery.

In one embodiment, the Vif-I compounds are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage, toxicity and therapeutic efficacy of such Vif-I compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies typically within a range of circulating concentrations that include the ED50 with little or no toxicity. The dosage can vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical compositions can be included in a container, pack, or dispenser together with instructions for administration.

The pharmaceutical compositions that include a Vif-I can be used in methods of treating a subject who is infected with a virus that comprises Vif, e.g., a lentivirus, e.g., HIV. The methods include administering a therapeutically effective amount of a Vif-I composition described herein to the subject, such that the viral load of the subject is reduced. As defined herein, a therapeutically effective amount of a Vif-I molecule is an amount sufficient to decrease HIV viral load in a subject infected with HIV. Methods for determining the viral load of a subject are known in the art. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the Vif-I compounds of the invention can include a single treatment or can include a series of treatments.

### EXAMPLES

The invention is further described in the following examples, which do not limit the scope of the invention described in the claims.

### Example 1

### Co-Expression of Vif and APOBEC3G Results in the Loss of APOBEC3G

In experiments originally designed to show interaction of Vif and APOBEC3G, it was observed that the total cellular levels of APOBEC3G were reduced dramatically in the presence of Vif.

The HIV-1 subgenomic proviral vector pNL-A1, which harbors HXB2 strain Vif, the corresponding pNL-A1 Δvif vector, and pNL-A1 C₁, which harbors the Vif^{C114S} mutant, were all generous gifts of Dr. Klaus Strebel, as described in Kao et al., (2003) J. Virol. 77:11398-11407. Single-cycle HIV-1 luciferase reporter virus pNL-Luc-E⁻R⁻ was used a source for NL4-3 strain Vif, and was a generous gift of Dr. Nathaniel Landau. A series of HIV-1 NL4-3 strain Vif deletion mutants, Δ2 (Δ12-23), Δ5 (Δ43-59), Δ6 (Δ58-74), Δ7 (Δ73-87), Δ9 (Δ97-112), Δ10 (Δ11-128), and Δ12 (Δ40-148) (as described in Simon et al., (1999) J. Virol. 73:2675-2681) were generous gifts of Dr. Michael Malim through Dr. David Kabat. All chemicals were purchased from Sigma (St. Louis, Mo.) unless otherwise indicated.

To generate Yellow Fluorescent Protein (YFP)-epitope tagged versions of Vif or Vif mutants, the Vif coding region was PCR amplified and cloned into the EcoR1 and BamH1 sites of pEYFP-C1 (BD Biosciences, Palo Alto, Calif.). The APOBEC3G coding region was amplified from cDNA derived from frozen human peripheral blood mononuclear cells (a generous gift from Dr. Mario Stevenson). To generate pCyan Fluorescent Protein (CFP)-APO, the APOBEC3G coding sequence was cloned into the HindIII and SacII sites of pECFP-C1 (BD Biosciences). For expression of APOBEC3G with a C-terminal epitope tag, a Kozak ribosome recognition sequence (ccacc) was placed directly upstream of the APOBEC3G start codon during PCR amplification. APOBEC3G with a C-terminal 3X hemagglutinin (HA) tag (pAPO-HA) and pAPO-CFP were engineered by cloning APOBEC3G into the EcoR1 and Xho1 sites of pIRES-hrGFP-2a (Stratagene, La Jolla, Calif.) and the HindIII and SacII sites of pECFP-N1 (BD Biosciences), respectively. Tat-Red Fluorescent Protein (RFP) was generated by cloning HIV-1 Tat into the HindIII and BamHI sites of pDsRED-N1 (BD Biosciences).

To induce random mutations within the Vif^{C114S} coding sequence, pNL-A1Vif^{C114S} was used as a template for low fidelity PCR and resulting products were cloned into pEYFP-C1, as described above. DNA from random colonies was prepared (Promega, Madison, Wis.) and used to transfect 293T cells.

293T cells were maintained in a humidified incubator (5% CO₂) at 37° C. in Dulbecco's modified Eagle's medium (DMEM; Invitrogen) supplemented with 10% fetal bovine serum, 100 units/ml penicillin and 100 μg/ml streptomycin (Invitrogen). Qiagen-purified plasmid DNA (Qiagen, Valencia, Calif.) was transfected into 293T cells using Lipofectaminem 2000 lipofection agent (Invitrogen). For Western blot and immunoprecipitation experiments, 293T cells were transfected in either 6 or 12 well plates when the cells were ˜60% confluent. The amount of vector used and the molar ratio of vector for co-transfection experiments are described in the Results and figure legends. When necessary, final DNA amounts were made equal by the addition of pGEM (Promega). For live imaging and immuno-localization, 293T cells were seeded into poly-d-lysine coated 35 mm glass bottom culture dishes and transfected when the cells were ˜50% confluent. For proteasome inhibition studies, culture media containing 100 μM of N-Acetyl-Leu-Leu-Nle-CHO (ALLN, Calbiochem, La Jolla, Calif.) or the equivalent volume of DMSO was added to cells 12 hours prior to harvesting or imaging 36 hours post-transfection.

293T cells were transfected with YFP-Vif and either CFP-APOBEC3G, APOBEC3G-HA, or HIV NL4 proviral DNA (wild type, which includes Vif, or a Vif-deficient strain) at an equimolar ratio (130 fmoles of each vector) using standard lipofection methods. Zero to twenty-four hours later, proteins were isolated and subjected to Western blot analysis. The blots were then analyzed using standard fluorometric methods.

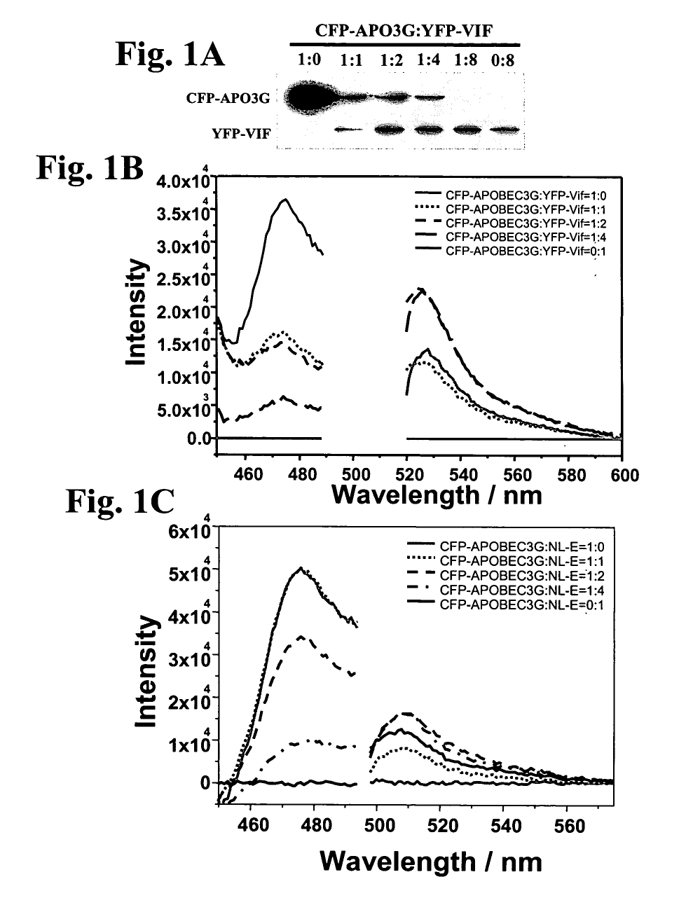

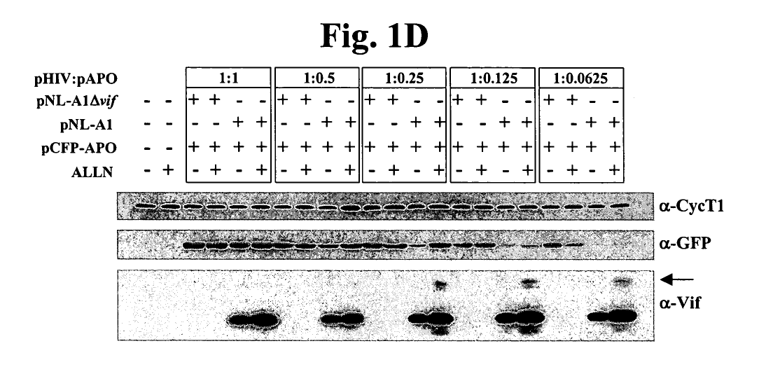

Co-expression of YFP-Vif with either CFP-APOBEC3G or APOBEC3G-HA at an equimolar ratio (130 fmoles of each vector) reduced the expression levels of both APOBEC3G fusion proteins (FIGS. 1A-B). Furthermore, expression of APOBEC3G was reduced to below detectable levels (by both Western and fluorometric analysis) with increasing amounts of YFP-Vif vector indicating that this effect was dose-dependent (FIGS. 1A-1B). This effect was specific to APOBEC3G, as the total cellular levels of endogenous CycT1 remained unaltered and CFP levels remained unaltered when co-expressed with YFP-Vif (FIG. 1A). The levels of CFP-APOBEC3G were also reduced when co-expressed with HIV NL4 wild-type proviral DNA, and not with a corresponding Vif-deficient strain, indicating the Vif was indeed responsible for this effect (FIG. 1C). Similar results were seen in HUT78 and HeLa cells.

The level of Vif-mediated depletion of APOBEC3G was further determined over a broad range of Vif (pNL-A1):APOBEC3G (pCFP-APO) vector ratios ranging from 1:1 to 1:0.0625, where 1 refers to 130 fmoles of vector. The level of depletion was determined by comparing the steady-state levels of CFP-APO when co-expressed with Vif to the equivalent Δvif control (pNL-A1Δvif). CFP-APO depletion by Vif was most significant between the 1:0.25 and 1:0.0625 ratios (FIG. 1D). Recovery of CFP-APO levels by proteasome inhibition (ALLN) was most dramatic at the 1:0.25 ratio (FIG. 1D). Despite significant depletion by Vif, only modest recovery of CFP-APO was observed at the 1:0.125 and 1:0.0625 ratios following proteasome inhibition (FIG. 1D). In the absence of Vif, CFP-APO levels were not significantly affected by proteasome inhibition except at the lowest level of pCFP-APO input (1:0.0625 ratio) for which a modest reduction in expression was observed (FIG. 1D). Identical expression profiles were also observed with pAPO-CFP or pAPO-HA in the presence and absence of Vif (data not shown). In the presence (FIG. 1D) or absence of CFP-APO, proteasome inhibition elevated the steady-state levels of Vif and resulted in the appearance of higher molecular weight species (FIG. 1D; arrow). The pNL-A1:pCFP-APO ratio of 1:0.25 was also visualized by confocal microscopy. In cells expressing Vif, CFP-APO was either not visible or was detected at a significantly reduced level relative to the Δvif control. Consistent with immuno-blot analysis, CFP-APO levels were elevated by proteasome inhibition and the number of cells visibly co-expressing the two proteins increased.

These results indicate that transient expression systems can be used to study the relationship between Vif and APOBEC3G effectively. Establishing the appropriate levels of expression is possible and may be crucial for studying the effects of Vif function on APOBEC3G.

### Example 2

### Subcellular Localization of Vif and APOBEC3G

The subcellular localization of Vif and APOBEC3G either expressed independently or together in live 293T cells was visualized by laser scanning confocal microscopy. YFP-Vif localized predominantly to the nucleus of live 293T cells. YFP-Vif was evenly distributed throughout the nucleus, but was excluded from nucleoli. Less intense cytoplasmic localization was also observed for YFP-Vif, suggesting that although predominantly nuclear, Vif is targeted to both compartments in 293T cells. Titrating the amount of transfected YFP-Vif vector showed that localization was observed first within the nucleus followed by more intense cytoplasmic staining concomitant with an increase in expression. This localization pattern was also observed with C-terminally labeled Vif. Furthermore, YFP-Vif expressed in the non-permissive HUT78 cell line exhibited both nuclear and cytoplasmic localization. CFP-APOBEC3G localized exclusively to the cytoplasm in live 293T cells. In the majority of cells observed, CFP-APOBEC3G appeared evenly distributed throughout the cytoplasm; however, a punctate and often perinuclear localization pattern was also observed. It remains unclear if this represents a functionally significant localization or a result of over-expression; however, this pattern was suggestive of co-localization with components of the secretory pathway and thus may have relevance to virion packaging. The localization patterns of either N-terminal or C-terminal labeled APOBEC3G were indistinguishable and the same localization pattern was observed in HUT78 cells. As observed in FIG. 1A-C, the total cellular levels of CFP-APOBEC3G were reduced when co-expressed with YFP-Vif and this effect appeared to be dose-dependent with no CFP-APOBEC3G detectable when co-expressed with 8-fold more YFP-Vif vector. Interestingly, YFP-Vif exhibited predominantly cytoplasmic localization when co-expressed with CFP-APOBEC3G, indicating that the mechanism by which Vif alters the expression levels of APOBEC3G occurs in the cytoplasm. This result suggests a functional interaction between Vif and APOBEC3G at the protein level as significant co-localization of the proteins was observed in the cytoplasm.

### Example 3

### Vif Does Not Target APOBEC3G for Proteasome Degradation

The results described in Example 2 suggested a functional interaction at the protein level and thus possible targeting of APOBEC3G for proteasome degradation. YFP-Vif and CFP-APOBEC3G were co-expressed at a 4:1 molar ratio of vectors (1=130 fmoles) in 293T cells and were treated with the proteasome inhibitors lactacystin (10 μM), ALLN (150 μM), or MG-132 (10 μM) 24 hours post-transfection.

To prepare total protein lysates, each well of a 6- or 12-well plate was washed once in phosphate buffered saline (PBS, Invitrogen) and then lysed in either 400 or 200 μl, respectively, of Mammalian Protein Extraction Reagent (M-PER, Pierce, Rockford, Ill.) supplemented with 0.5% (v/v) Triton-X 100 (Pierce), 150 mM NaCl, 5 mM EDTA, and a 1/100 (v/v) dilution of a protease inhibitor cocktail for mammalian tissue for 30 minutes at 4° C. with gentle rotation. Lysates were harvested from the well and insoluble material was removed by centrifugation for 5 minutes at full-speed in a microcentrifuge. Protein concentration was determined by D_{c} protein assay (Bio-Rad, Hercules, Calif.). For immunoprecipitation, 0.5 mg of lysate was diluted to 0.5 mg/ml in 1 ml of lysis buffer. APO-HA was precipitated by incubation with agarose-conjugated rabbit α-HA (20 μg IgG; Santa Cruz Biotechnology, Inc., Santa Cruz, Calif.). To immunoprecipitate CFP-APO, lysates were first pre-cleared with a 50 μl bed volume of Protein G Sepharose (Amersham Pharmacia Biotech, Piscataway, N.J.) for 1 hour at 4° C. CFP-APO was then precipitated from pre-cleared lysates by incubation with 5 μg of a α-GFP rabbit polyclonal (BD Biosciences) for 3 hours at 4° C. Antibody was captured by incubation with a 50 μl bed volume of Protein G Sepharose for 1 hour at 4° C. followed by 4 washes in 1 ml of lysis buffer for 10 minutes each time. Protein was eluted by boiling for 5 minutes at 100° C. in sample buffer [50 mM Tris-HCI, pH 6.8, 100 mM dithiothreitol, 2% (w/v) SDS, 0.1% (w/v) bromophenol blue, 10% (v/v) glycerol].

For SDS-PAGE of protein lysates, samples were denatured and reduced by adding 4× SDS-PAGE sample buffer followed by boiling at 100° C. for 5 min. Protein was resolved by 12% SDS-PAGE and transferred onto a polyvinylidene difluoride membrane (PVDF, Bio-Rad Laboratories, Inc., Hercules, Calif.) using a Semi-Dry Electroblotter (Bio-Rad). Following transfer, membrane was blocked overnight in 5% (w/v) nonfat dry milk in TBS-T [20 mM Tris, pH 7.4, 150 mM NaCl, 0.1% (v/v) Tween 20] and washed 3× for 10 minutes each in TBS-T before and after the addition of antibody. All antibodies were diluted in 2.5% (w/v) nonfat dry milk in TBS-T. CFP, YFP, and GFP were detected using a mouse monoclonal antibody (MAb) against GFP diluted to 1 μg/ml (BD Bioscience). RFP was detected with a rabbit polyclonal (BD Biosciences) diluted to 0.1 μg/ml. Human CycT1 was detected with a goat anti-CycT1 polyclonal antibody (Santa Cruz Biotechnology) diluted to 0.1 μg/ml. HA was detected with a rabbit polyclonal antibody (Santa Cruz Biotechnology, Inc.) diluted to 0.02 μg/ml. Vif and Vif mutants were detected using a Vif MAb diluted 1/5000 (this reagent was obtained through the AIDS Research and Reference Reagent Program, Division of AIDS, NIAID, NIH: HIV-1 Vif Monoclonal Antibody (#319) from Dr. Michael H. Malim (see Simon et al., (1995) J. Virol. 69:4166-4172; Simon et al., (1997) J. Virol. 71:5259-5267; and Fouchier et al., (1996) J. Virol. 70:8263-8269)). All horseradish peroxidase-conjugated secondary antibodies were used at a dilution of 0.05 μg/ml (Santa Cruz Biotechnology, Inc.). Blots were developed with the BM Chemiluminescence Blotting Kit (Roche Molecular Biochemicals, Indianapolis, Ind.) and exposed to Kodak BioMax MR X-ray film (Eastman Kodak Company, Rochester, N.Y.).

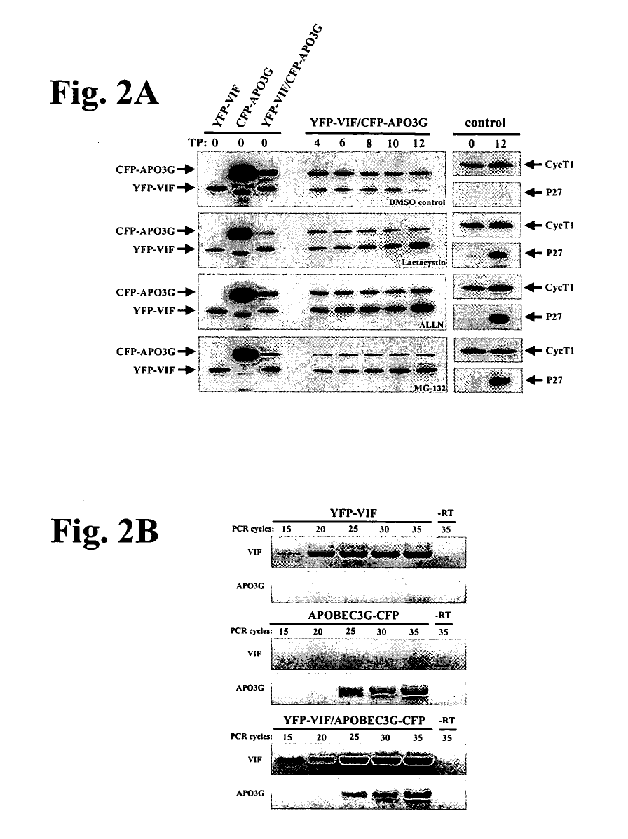

Addition of inhibitor represents time-point 0 and total cell lysates were made at two hour intervals beginning at 4 hours and ending at 12 hours post-addition of proteasome inhibitor. As a positive control for proteasome inhibition, the endogenous levels of the cyclin-dependent kinase inhibitor, p27, a protein known to be targeted for proteasome degradation was observed before (TP 0) and after (TP 12) the 12 hour time course with inhibitors. At the total cell protein level loaded per lane, p27 was barely detectable; however, the levels of the protein rose dramatically after 12 hour incubation with all three proteasome inhibitors. As a control for protein loading, the endogenous levels of CycT1 were observed on the same blot. Results are shown in FIG. 2A. As expected, the levels of CFP-APOBEC3G were reduced in the presence of Vif; however, proteasome inhibition by all three inhibitors resulted in no obvious effect on CFP-APOBEC3G expression. Conversely, the levels of YFP-Vif appeared to increase with proteasome inhibition suggesting that a subset of YFP-Vif may be degraded through the proteasome. This may not be altogether surprising as FIG. 1A showed that the levels of Vif appear to plateau. These results do not exclude the possibility that Vif mediates degradation of APOBEC3G through a proteasome-independent pathway.

### Example 4

### Vif Does Not Mediate Degradation of APOBEC3G mRNA

To determine if Vif mediates degradation of APOBEC3G mRNA the total cellular levels of message were observed by real-time PCR. Total cellular RNA was isolated using standard methodology from cells expressing YFP-Vif alone, CFP-APOBEC3G alone, or both at an 8:1 molar ratio (1=130 fmoles) of transfected vector, respectively, and reverse transcribed using oligo d(T) primer to specifically detect mRNA. No obvious difference was observed in the total cellular levels of either YFP-Vif or CFP-APOBEC3G mRNA when co-expressed (FIG. 2B). This experiment rules out Vif functioning on the levels of transcription or mRNA processing and suggests that Vif does not function at the level of mRNA degradation or turnover. Furthermore, this confirmed that both expression vectors were successfully co-transfected, demonstrating that the decrease in CFP-APOBEC3G expression was not due to a phenomena associated with co-transfection.

### Example 5

### Vif Inhibits APOBEC3G mRNA Translation, But Not Export

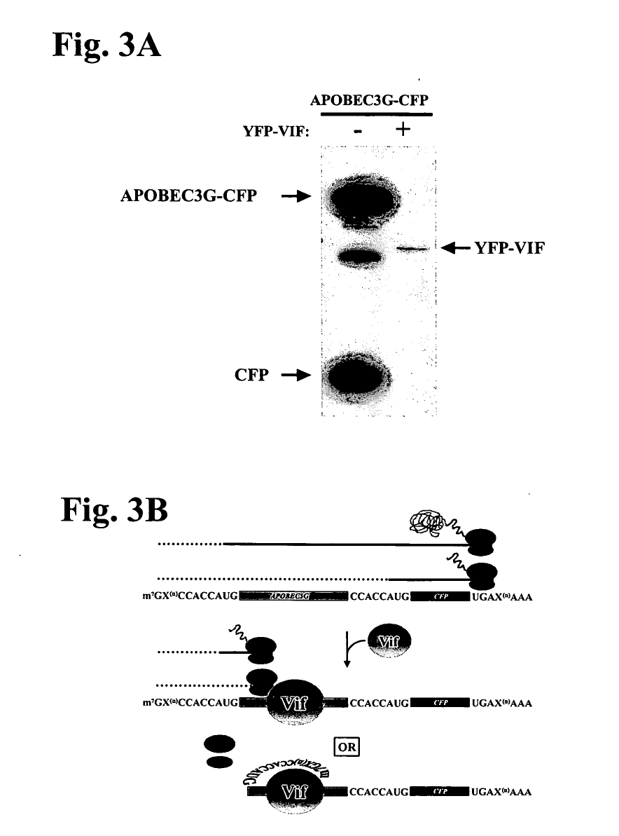

Considering that CFP-APOBEC3G was not degraded via the proteasome pathway and that total mRNA levels of APOBEC3G appeared unaltered in the presence of YFP-Vif, we postulated that Vif may function at the levels of either mRNA export or inhibition of translation. Since expression of CFP-APOBEC3G results in an increase in YFP-Vif localization to the cytoplasm, the latter mechanism of inhibition seems most likely. To test this, an expression vector was engineered to express independent proteins from the same transcript through the use of multiple start codons with Kozak sequences to facilitate translation initiation. APOBEC3G was cloned directly upstream of CFP which already harbored its own start codon and Kozak sequence (CCACC) to facilitate translation initiation. An additional Kozak sequence was placed directly upstream of the APOBEC3G start codon (FIG. 3B), and it was shown by Western blot analysis of total cell lysates that expression from this vector after 24 hours in 293T cells resulted in the translation of both APOBEC3G-CFP and CFP alone (FIG. 3A). When co-expressed with YFP-Vif, the expression of both APOBEC3G-CFP and CFP was reduced.

These results showed that YFP-Vif was affecting the translation of both APOBEC3G-CFP and CFP from the same APOBEC3G-CFP transcript, and that YFP-Vif was specifically inhibiting translation of APOBEC3G-CFP mRNA regardless of the translation start site. Thus, collectively, this analysis strongly suggested that Vif specifically functions to alter APOBEC3G expression by inhibiting translation and that the elements required for this inhibition resided within the mRNA sequence encoding APOBEC3G. When co-expressed with YFP-Vif, the expression of both APOBEC3G-CFP and CFP was reduced indicating that Vif functions by preventing translation. Based on these results, we propose a model in which Vif binds to region(s) within the APOBEC3G mRNA coding sequence, thus either preventing translation elongation or ribosome binding (FIG. 3B). It is not clear whether this event initiates within the nucleus and then Vif is transported out of the nucleus with APOBEC3G mRNA or cytosolic synthesized Vif binds to cytosolic mRNA preventing normal Vif localization to the nucleus.

### Example 6

### High Throughput Screening of a Small Molecule Library

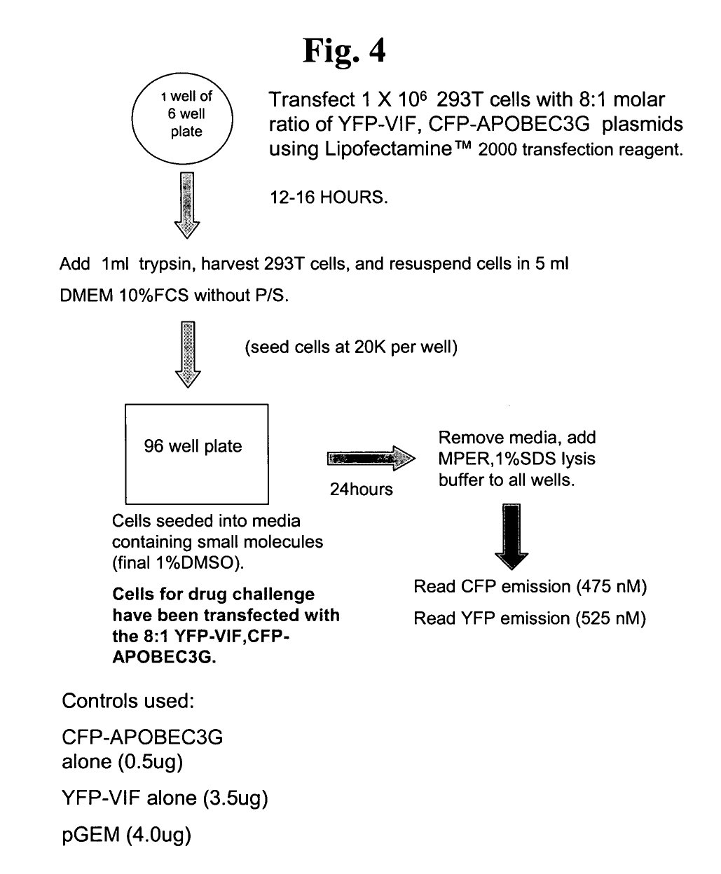

The compounds in a small molecule library were screened for the ability to affect Vif-mediated reduction in APOBEC3G protein levels. The protocol is illustrated in FIG. 4. Briefly, 1×10⁶ 293T cells in each well of a 6 well plate were transfected with an 8:1 molar ratio of YFP-Vif and CFP-APOBEC3G encoding plasmids using Lipofectamine 2000™ transfection reagent. The cells were cultured for 12-16 hours, then 1 ml of trypsin was added to the cells to harvest them. The cells were resuspended in 5 ml DMEM 10% FCS without penicillin/streptomycin. The cells were then seeded into media containing the small molecules of the library in the wells of a 96 well plate, with a final concentration of 1% DMSO. After culture for 24 hours, the media is removed and MPER with 1% SDS lysis buffer is added to all of the wells. Fluorescence emission was read using a plate reader (Tecan, Maennedorf, Switzerland) as follows: CFP emission (475 nM); YFP emission (525 nM). Cells transfected with CFP-APOBEC3G alone (0.5 μg); YFP-Vif alone (3.5 μg); or empty pGEM (4.0 μg) were used as controls.

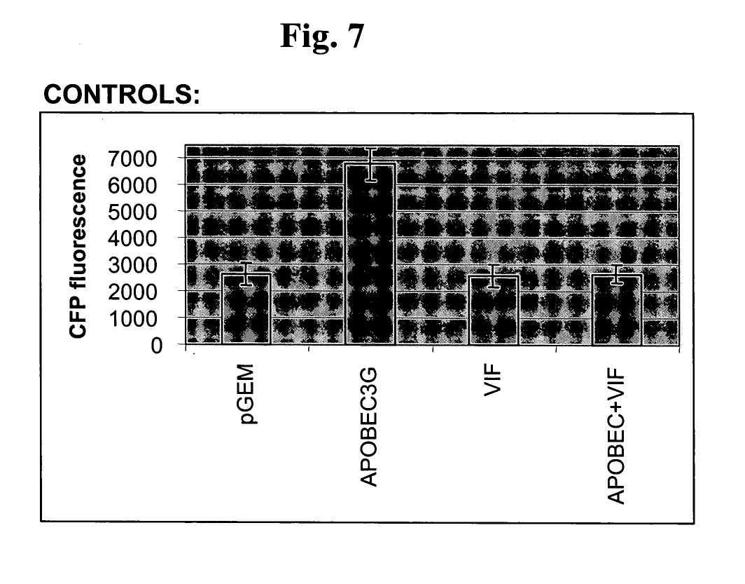

The results are shown in FIG. 7, which represents the CFP fluorescence measured in well containing cells transfected with pGEM, CFP-APOBEC3G, YFP-Vif, or CFP-APOBEC3G and YFP-Vif. As expected, expression of pGEM or Vif-YFP resulted in negligible fluorescent emission at 475 nm. Expression of CFP-APOBEC3G resulted in significant fluorescence at 475 nm, and this fluorescence was substantially reduced by the co-expression of YFP-Vif.

Using this system, a combinatorial library of small molecular compounds was screened for Vif-inhibitory activity. Several compounds were identified that demonstrated the ability to rescue APOBEC3G expression in the presence of Vif; these compounds are illustrated in FIG. 8.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying an modulator of an activity of a first protein, wherein the activity of the first protein modulates a level of a second protein, the method comprising:
obtaining a sample comprising a cell expressing a first tagged protein and a second tagged protein;
contacting the sample with a test compound;
determining a level of a first protein and a level of a second protein in the presence of the test compound;
determining a test ratio of the levels of the first and second proteins in the presence of the test compound; and
obtaining a reference ratio of a level of the first and second proteins in the absence of the test compound, wherein a change in the test ratio compared to the reference ration indicates that the test compound is a modulator of the activity of the first protein.

2. The method of claim 1, wherein the activity of the first protein causes a reduction in the level of the second protein.

3. The method of claim 1, wherein the activity of the first protein causes an increase in the level of the second protein.

4. The method of claim 1, wherein the first protein acts by affecting one or more of transcription, translation, sub-cellular localization, degradation, or post-translational modification of the second protein.

5. The method of claim 1, wherein the first and second tagged proteins each comprise fluorescent tags that are excited at different wavelengths, emit at different wavelengths, or both.

6. The method of claim 5, wherein the fluorescent tags are selected from the group consisting of green fluorescent protein, yellow fluorescent protein, red fluorescent protein, cyan fluorescent protein, Kindling red protein, and JRed.

7. The method of claim 1, wherein the cell further expresses a third tagged protein.

8. The method of claim 7, wherein the first, second, and third tagged proteins each comprise fluorescent tags that are excited at different wavelengths, emit at different wavelengths, or both.

9. The method of claim 1, wherein the first protein is Virion Infectivity Factor (Vif) and the second protein is apolipoprotein B mRNA-editing enzyme, catalytic polypeptide-like 3G (APOBEC3G).

10. The method of claim 1, wherein the first protein is p53 and the second protein is selected from the group consisting of cyclin-dependent kinase inhibitor p21 WAF-1, 14-3-3, reprimo, bax, Death receptor 5 (DR5), p53-regulated apoptosis-inducing protein 1 (p53AIP), p53 Protein Induced with Death Domain (PIDD), NOXA, p53 Upregulated Modulator of Apoptosis (PUMA), Fas/Apoptosis Inducing Protein 1 (Fas/APO-1), and by redox related proteins.

11. The method of claim 1, wherein the first protein is Mdm2 and the second protein is p53.

12. A small molecule inhibitor identified by the method of claim 9.

13. A composition comprising a small molecule inhibitor of Virion Infectivity Factor (Vif) and a pharmaceutically acceptable carrier.

14. The composition of claim 13, wherein the small molecule inhibitor has a formula of Formula I: 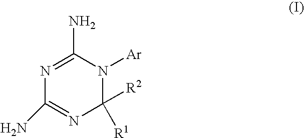
wherein
R¹ is C₁-C₆ alkyl, alkenyl, alkynyl, cycloalkyl, or cycloalkenyl, R¹ being optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group;
R² is C₁-C₆ alkyl, alkenyl, alkynyl, cycloalkyl, or cycloalkenyl, R² being optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group;
Ar is an aryl or heteroaryl group optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group; and
each R^{a}, R^{b}, and R^{c} is, independently, hydrogen, halo, substituted or unsubstituted amino, or a substituted or unsubstituted C₁-C₆ acyl, alkyl, alkenyl, alkynyl, cycloalkyl or cycloalkenyl group.

15. The composition of claim 13, wherein the small molecule inhibitor has a formula of Formula II 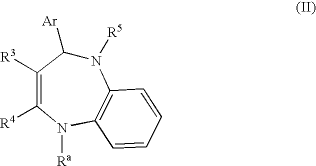
wherein
R³ and R⁴ are each, independently, C₁-C₆ alkyl, alkenyl, alkynyl, cycloalkyl or cycloalkenyl, or R³ and R⁴ together are a C₂-C₆ alkyl or alkenyl chain;
R³ and R⁴ are, independently or together, optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group;
Ar is an aryl or heteroaryl group optionally substituted with one or more of: —R^{a}, —OR^{a}, ═O, —SR^{a}, ═S, —C(═O)NR^{a}R^{b}, —NR^{a}R^{b}C(═O)R^{c}, or an aryl group;
R⁵ has the formula —C(═O)R^{a}; and
each R^{a}, R^{b}, and R^{c} is, independently, hydrogen, halo, substituted or unsubstituted amino, or a substituted or unsubstituted C₁-C₆ acyl, alkyl, alkenyl, alkynyl, cycloalkyl or cycloalkenyl group.

16. The composition of claim 13, wherein the inhibitor is a phenyl dihydrotriazine

17. The composition of claim 16, wherein the phenyl dihydrotriazine is HK-I-5 or HK-I-27 as shown in FIG. 8.

18. The composition of claim 16, wherein the phenyl dihydrotriazine is 4,6-diamino-1-(3′-methylphenyl)-1,2-dihydro-2,2-dimethyl-s-triazine hydrochloride or 4,6-diamino-1-(2′,6′-dibromo-4′-methylphenyl)-1,2-dihydro-2,2-dimethyl-s-triazine hydrochloride as shown in FIG. 8.

19. The composition of claim 13, wherein the inhibitor is a benzodiazepan.

20. The composition of claim 19, wherein the benzodiazepan is TR0383 as shown in FIG. 8.

21. The composition of claim 19, wherein the benzodiazepan is cyclopropyl-(4-phenyl-1H-benzo[b][1,4]diazepin-5(4H)-yl)methanone as shown in FIG. 8.

22. A method of treating a subject infected with a virus, the method comprising administering to the subject a therapeutically effective amount of a composition of claim 13.

23. The method of claim 22, wherein the subject is infected with HIV-1.
